(19) United States
(12) Patent Application Publication
(10) Pub. No.: US 2021/0145471 A1
(43) Pub. Date: May 20, 2021

(54) MECHANICAL RESECTION BLADE

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(72) Inventor: Rafal Z. JEZIERSKI, San Francisco, CA (US)

(73) Assignees: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(21) Appl. No.: 17/093,867

(22) Filed: Nov. 10, 2020

Related U.S. Application Data
(60) Provisional application No. 62/938,068, filed on Nov. 20, 2019.

(51) Int. Cl. A61B 17/32

(52) U.S. Cl. CPC A61B 17/32002; A61B 2217/005; A61B 2217/007

(57) ABSTRACT

Embodiments of the invention include a cutting tool with an outer component having a window and an inner component sized to fit within the outer component and be rotated relative to the outer component. The outer component window may be shaped to improve cutting effectiveness and efficiency of the device. Some embodiments of the invention also include one or more of drive units, resection controls, and fluid management systems. Embodiments of the invention may also include methods of using the cutting tool and related systems.

### CROSS-REFERENCE TO RELATED APPLICATIONS

This application claims the benefit of U.S. Provisional Application Ser. No. 62/938,068 filed Nov. 20, 2019 titled “Mechanical Resection Blade.” The provisional application is incorporated by reference herein as if reproduced in full below.

### FIELD OF THE INVENTION

The present invention relates generally to the field of surgical instruments, and more particularly relates to surgical instruments such as cutting mechanisms typically used in endoscopic or arthroscopic surgery to alter tissue. Some embodiments include improved blade configurations that are capable of more effectively altering desired tissue components.

### BACKGROUND

Surgical cutting or resection devices with drive units and rotating blades, also referred to as cutting elements, are commonplace in endoscopic and arthroscopic surgery. Inner and the outer cutting elements may be formed from cylindrical tubular members that have been altered to include cutting surfaces. A distal end of one or both of the inner and outer cutting elements are disclosed in the prior art as rounded, for example in a spherical shape or a “bullet” shape. A bullet-shaped distal end may be superior to a spherically-shaped distal end in some applications. It is also known in the prior art to remove a portion of an outer cutting element, bullet-shaped distal end diagonally along a straight line to expose a portion of the inner cutting element when the inner cutting element is rotated relative to the outer cutting element. However, a straight-line exposure on a bullet-shaped tip provides a somewhat narrow window in the outer cutting element through which it may be difficult to effectively and efficiently cut or shape tissue.

Therefore, a challenge to be overcome is to provide for effective and efficient cutting of tissue through a window in an outer cutting element that has been made in a bullet-shaped distal end of the outer cutting element. An improved device and method may provide for both greater cutting width and rate of tissue removal without expanding the maximum diameter of the resection device. Improved results have been demonstrated with devices and methods disclosed herein. Specifically, testing of at least one embodiment of an improved cutting tool with a bullet-shaped tip have shown improvements in outer component window size area of over 40% for an identically sized outer component. Improvements where achieved by modifying the shape of the window in the outer component from a straight cut to a concave cut. Increases in the resection rate of improved devices have also been demonstrated.

### SUMMARY

An embodiment of the invention is a cutting tool with an outer component and an inner component. The outer component has a longitudinal axis and includes a cylindrical portion, an ogive-shaped distal portion coupled to the cylindrical portion, and an outer component window into a central hollow portion in the outer component. The inner component is sized to fit within the central hollow portion of the outer component and be rotated relative to the outer component. In some embodiments, the outer component window at least in part includes a curvature relative to the longitudinal axis of the outer component.

Another embodiment of the invention is a system for cutting that includes a cutting tool, a resection control, and a fluid management system. The cutting tool may include an outer component having a longitudinal axis, the outer component including a cylindrical portion, an ogive-shaped distal portion coupled to the cylindrical portion, and an outer component window into a central hollow portion in the outer component. Embodiments of the cutting tool also include an inner component sized to fit within the central hollow portion of the outer component and be rotated relative to the outer component, and a drive unit coupled to the outer component and coupled to the inner component and capable of rotating the inner component relative to the outer component. In some embodiments, the outer component window at least in part includes a curvature relative to the longitudinal axis of the outer component. The resection control may be electrically coupled to the cutting tool, and the resection control may be configured to provide control signals to the cutting tool. Fluid management system embodiments include a pump control and a fluid passage between the pump control and the surgical site. The fluid management system is configured to control a fluid supply to the surgical site and may include suction through the cutting tool back to the pump control.

Yet another embodiment of the invention is a method of removing tissue with a system for cutting. Such a method may include advancing a cutting tool with an outer component having a bullet-shaped distal end and an outer component window into tissue. The outer component window may at least in part include a curvature relative to the longitudinal axis of the outer component. The method may also include injecting fluid into a surgical site and suctioning fluid through the cutting tool under control of a fluid management system. Method embodiments may also include rotating an inner component sized to fit within a central hollow portion of the outer component relative to the outer component under control of a resection control such that a portion of the inner component intermittently extends through the outer component window to shear and remove tissue that is adjacent to the cutting tool.

### BRIEF DESCRIPTION OF THE DRAWINGS

FIG. 1 is a perspective view of an embodiment of a cutting tool.

FIG. 2 is side elevation view of the distal end of the cutting tool of FIG. 1.

FIG. 3 is a side elevation view of an embodiment of the inner component of the cutting tool of FIG. 2.

FIG. 4 is a side elevation view of the inner component illustrated in FIG. 3 that has been rotated about its longitudinal axis to show the lowest vertical profile of the inner component.

FIG. 5 is a side elevation view of an alternate embodiment of the inner component of the cutting tool.

FIG. 6 is a side elevation view of the outer component of the cutting tool of FIG. 2 illustrating a radius of the concave outer component window and comparing the outer component window to a straight line “SL” outer component window.

FIG. 7 is another embodiment of an outer component with a window that has a concave proximal shape and a relatively smaller concave distal shape.

FIG. 8 is another embodiment of an outer component with a window that has a concave proximal shape and a similarly sized convex distal shape.

FIG. 9 is another embodiment of an outer component with a window that has a concave proximal shape and a relatively smaller convex distal shape.

FIG. 10 is another embodiment of an outer component with a window that has a concave proximal shape and a relatively larger convex distal shape.

FIG. 11 is another embodiment of an outer component with a window that has a convex proximal shape and a relatively smaller concave distal shape.

FIG. 12 is another embodiment of an outer component with a window that has a convex proximal shape and similarly sized concave distal shape.

FIG. 13 is another embodiment of an outer component with a window that has a convex proximal shape and relatively larger concave distal shape.

FIG. 14 is a system diagram of the cutting tool of FIG. 1 in combination with a resection control and fluid management system for controlling use of the cutting tool and supplying and discharging fluids.

### DETAILED DESCRIPTION

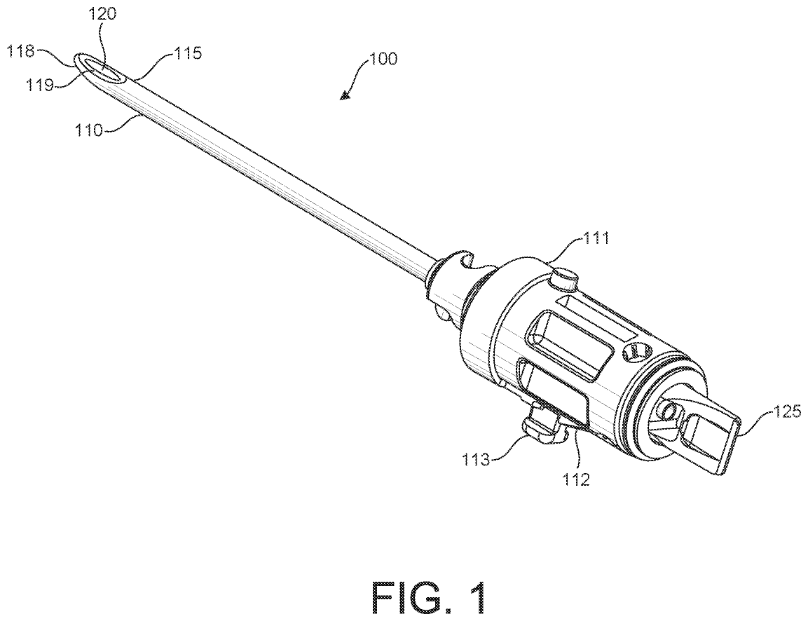

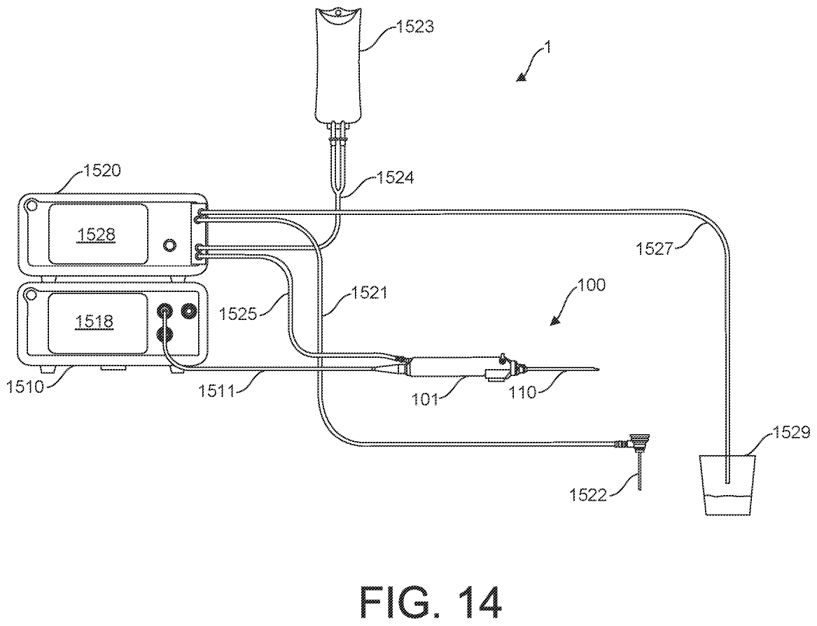

A system for cutting 1 with a cutting tool 100 and respective component parts are illustrated in FIGS. 1-14. As used herein the term “cutting tool” may include not only tools that cut with a blade but tools that abrade, scratch, rub, dislodge, or otherwise manipulate tissue. The cutting tool 100 illustrated includes a drive unit 101 (FIG. 14), an outer component 110, 210, 310, 410, 510, 610, 710, 810 (FIGS. 1, 2, and 6-14), and an inner component 120, 220 (FIGS. 1-5). The drive unit 101 may be a motorized drive unit powered by an electric motor and a battery, transformer, capacitor, wire, or other source of electricity, may be powered by air pressure or other fluid pressure, may be powered by manual or automated user manipulation, or may be powered by any other effective mechanism. The drive unit 101 may include any effective set controls for dictating the function of the drive unit 101. The set of controls may include buttons, switches, sliders, indicators, and other mechanisms or displays to adjust and control functions of the drive unit 101. For example and without limitation, the controls may be used to one or more of power the drive unit 101 on and off, set a rotating speed for a portion of the drive unit, activate a clockwise or counterclockwise rotation of a portion of the drive unit, indicate a status or function of the drive unit 101, and provide any other useful control or display associated with the drive unit 101. Function and control may also be accomplished by use of a control system coupled with the drive unit 101 and may further include use of wired or wirelessly connected remote controls such as foot operated controls.

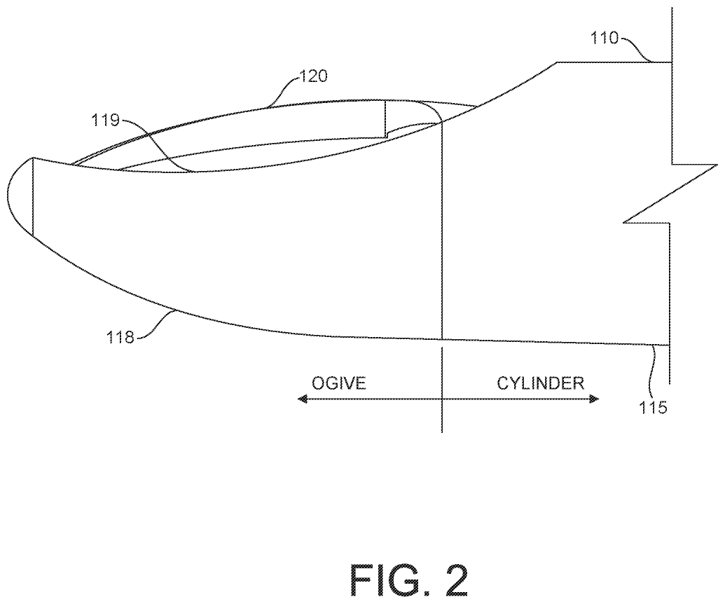

The illustrated outer component 110 (FIGS. 1, 2, 6, and 14) has a longitudinal axis that extends between its proximal and distal ends substantially at the cross-sectional center of the outer component 110. The outer component 110 depicted includes a cylindrical portion 115, an ogive-shaped distal portion 118 coupled to the cylindrical portion 115, and an outer component window 119 into a central hollow portion in the outer component 110. See FIG. 2 for a specific illustration of locations of the ogive-shaped distal portion 118 and the cylindrical portion 115. As used herein, an “ogive” or “ogive-shaped” portion means a pointed, curved surface such as is often employed to form an approximately streamlined nose of a penetrating body, bullet, or projectile. As used herein an ogive is not merely a simple spherical tip formed at the distal end of a cylindrical section or tube.

Following is a non-limiting list of shapes that fit these parameters for an ogive and are consider an ogive as used herein: a secant ogive with a circular arc of greater radius than the diameter of a cylindrical section or tube to which it is attached, as drawn from the edge of the tube until it intercepts a central axis of the tube; a spherically blunted secant ogive with a secant ogive with a distal tip that has been rounded over; a tangent or spitzer ogive with a circular arc drawn so that it meets the shank at an angle of zero, i.e., the distance of the center of the arc from a central axis, plus the radius of the shank, equals the radius of the arc; a spherically blunted tangent ogive with a tangent ogive with a distal tip that has been rounded over; an elliptical ogive with a curve very similar to the spitzer ogive, except that the circular arc is replaced by an ellipse defined in such a way that it meets the central axis at exactly 90° (this gives a somewhat rounded nose regardless of the sharpness ratio); a conic ogive; a spherically blunted conic ogive; a bi-conic ogive; a spherically blunted bi-conic ogive; parabolic series ogive shapes, which are generated by rotating a segment of a parabola around a line parallel to its latus rectum (similar to that of the tangent ogive, except that a parabola is the defining shape rather than a circle), and associated spherically blunted parabolic series ogive shapes; power series ogives (including the shape commonly referred to as “parabolic”, but power series is more general) characterized by a blunt tip, and by the fact that its base is not tangent to the body tube (the shape may be modified at the base to smooth out this discontinuity); and Haack series ogives—typically mathematically derived for the purpose of minimizing drag, not constructed from geometric figures, including but not limited to: LD-Haack [Von Kaman] with a C value of zero, LV-Haack with a C value of ⅓, and Tangent with a C value of ⅔.

The outer component 110 illustrated includes a base 111 (FIG. 1) near a proximal end of the outer component 110. The base 111 depicted is configured to releasably lock with the drive unit 101 through the latch 112 (FIG. 1). Insertion of the base 111 into the drive unit 101 leads to engagement of the latch 112 with the drive unit 101 (FIG. 14). The latch 112 may be released by pressing the adjacent guide pin 113. While pressed, the base 111 may be removed from the drive unit 101.

The illustrated cylindrical portion 115 is coupled to a distal end of the base 111. In the illustrated embodiment, the central hollow portion of the outer component 110 is substantially round. In other embodiments, the central hollow portion may be formed in another shape to interact with an adjacent component, such as a bearing, that has a complementary shape. For example and without limitation, a central hollow portion may have angular features that fit with angular features of an outer component to lock the bearing relative to the outer component. The outer component 110 depicted includes a closed distal tip at a cross-sectional center of the outer component 110. Other embodiments may include distal tips that are open or in some part more open than the illustrated embodiment.

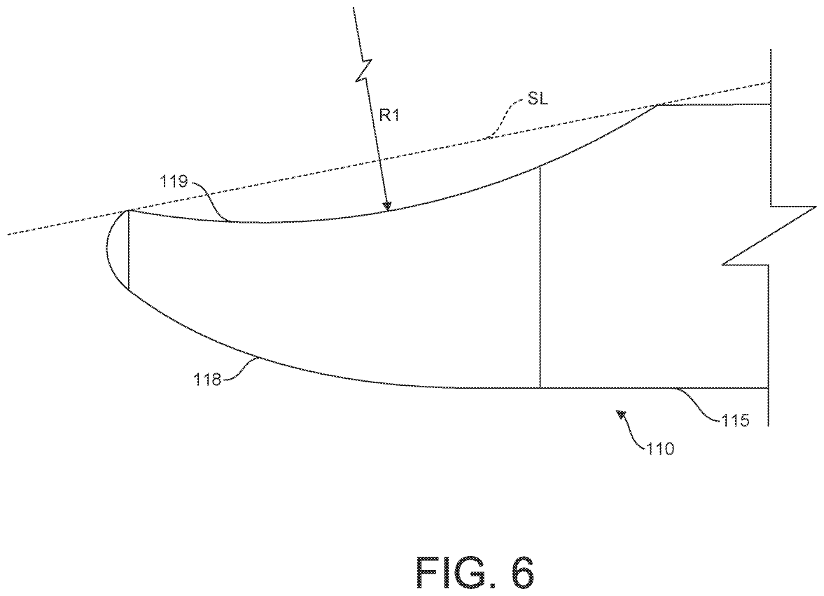

The outer component window 119 (FIGS. 1, 2, and 6) at least in part includes a curvature relative to the longitudinal axis of the outer component 110. This curvature is in contrast to a straight line window, as is illustrated in FIG. 6 with dashed line SL. A straight line window opening is a known configuration compared to which curved outer component windows have some superior attributes. As used herein, a curvature of a window having a curve relative to the longitudinal axis of the outer component means that the curve diverges or converges from the longitudinal axis at a non-constant rate. For example, the outer component window 119 is shown in FIG. 6 with a radius R1 diverges and converges from the longitudinal axis at a non-constant rate. The radius R1 from its distal extent converges toward the longitudinal axis of the outer component and then nearer the proximal extent of the window diverges from the longitudinal axis of the outer component. Note that from a location not on the plane of a straight line cut, the window created may appear to be curved relative to the longitudinal axis of the outer component, but the fact that the cut itself is not made along a curve relative to the longitudinal axis of the outer component means that the configuration is not within the definition of a window having a curve relative to the longitudinal axis of the outer component, as used herein. The outer component window 119 depicted is located above the longitudinal axis of the outer component 110. In other embodiments, some of the outer component window may intersect with the longitudinal axis of the outer component. In the illustrated embodiment, a proximal end of the outer component window 119 is between about 0.3 inches and 0.4 inches from the distal tip of the outer component 110. Other embodiments may include other outer component window lengths, but the illustrated length has been found to be effective. The radius R1 may be between about 1.5 inches and 3.0 inches in some embodiments. In some embodiments, the radius of the outer component window is a precise radius of a fixed amount, but in other embodiments, the radius may be somewhat varied along its length, for example to transition between two or more radii within a range. Changing the radius R1 to various degrees, in combination with various styles and sizes of inner components, will calibrate the aggressiveness and type of cut made by the cutting tool 100. The outer component window 119 curvature defined by the radius R1 in FIGS. 2 and 6 passes through both the ogive-shaped distal portion 118 and the cylindrical portion 115 of the outer component 110. In other embodiments, the outer component window may only pass through ogive-shaped distal portion.

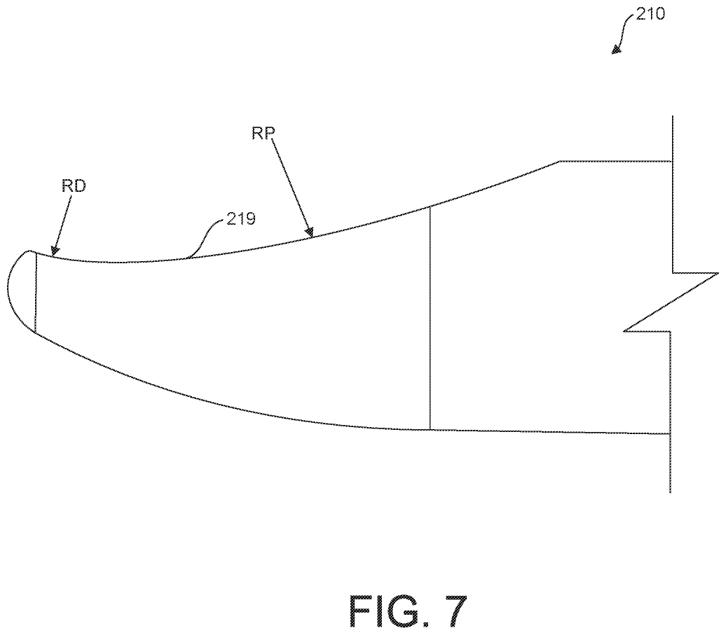

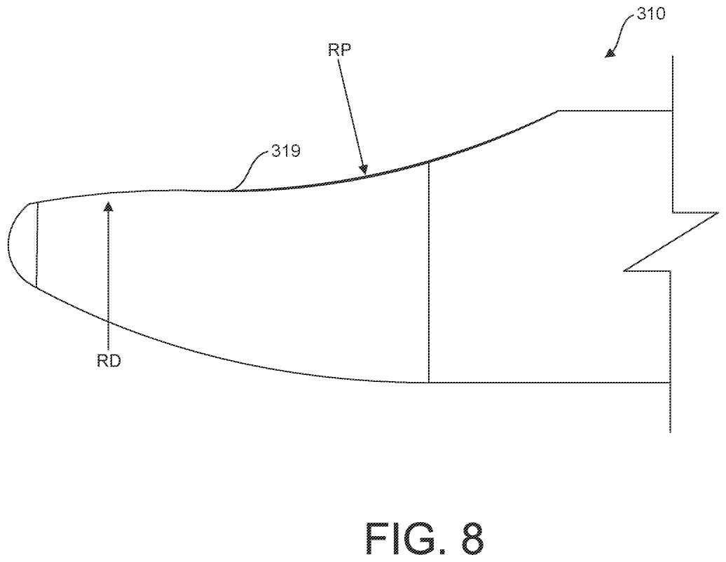

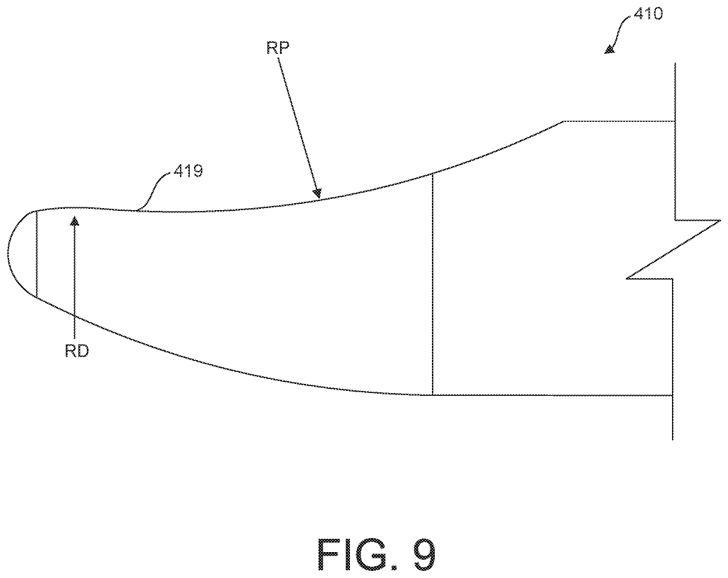

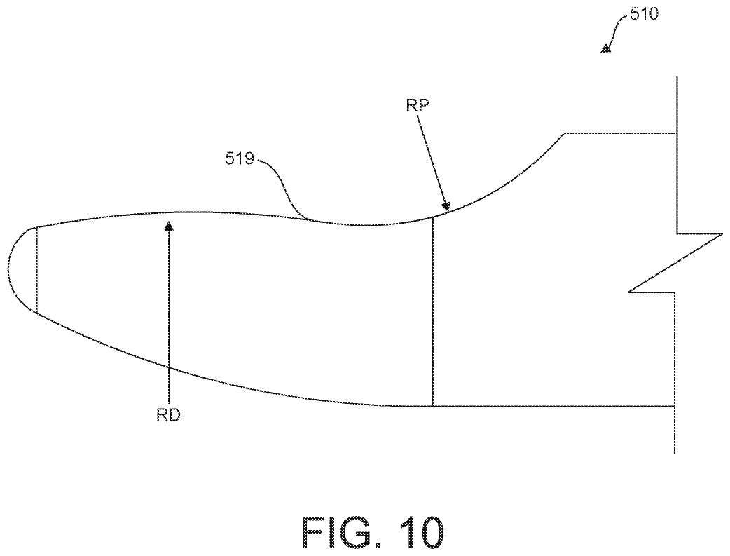

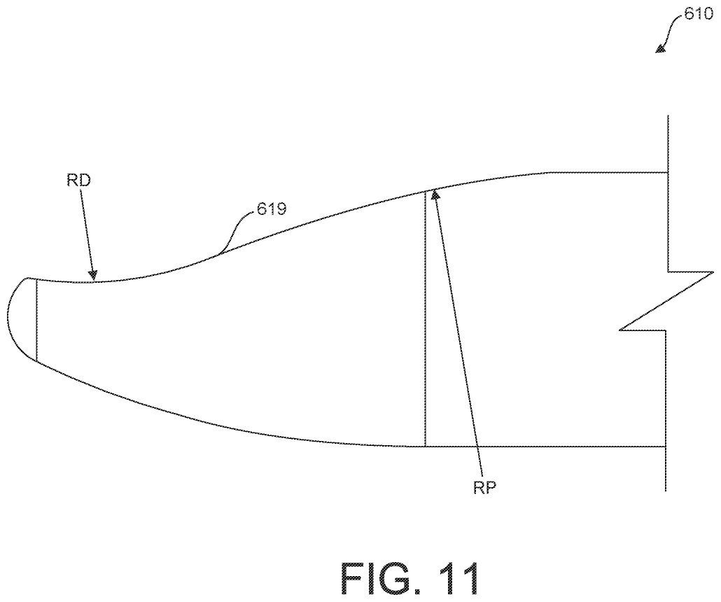

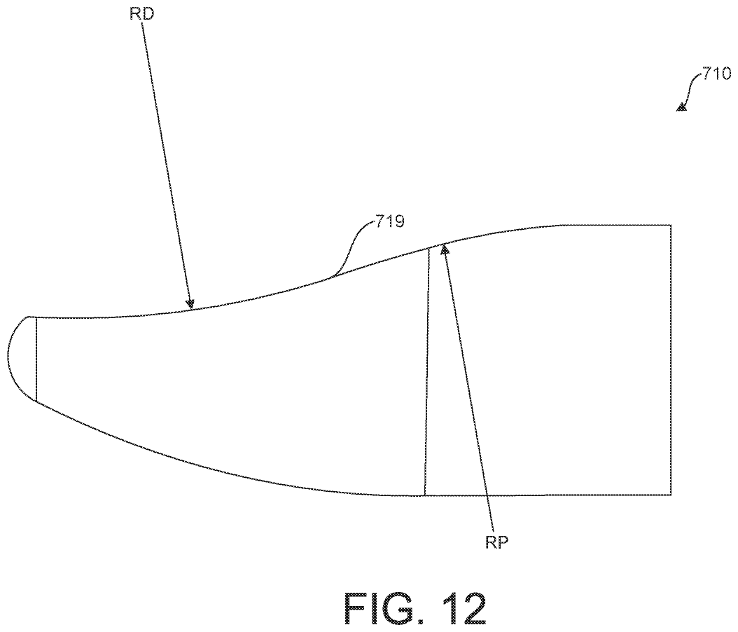

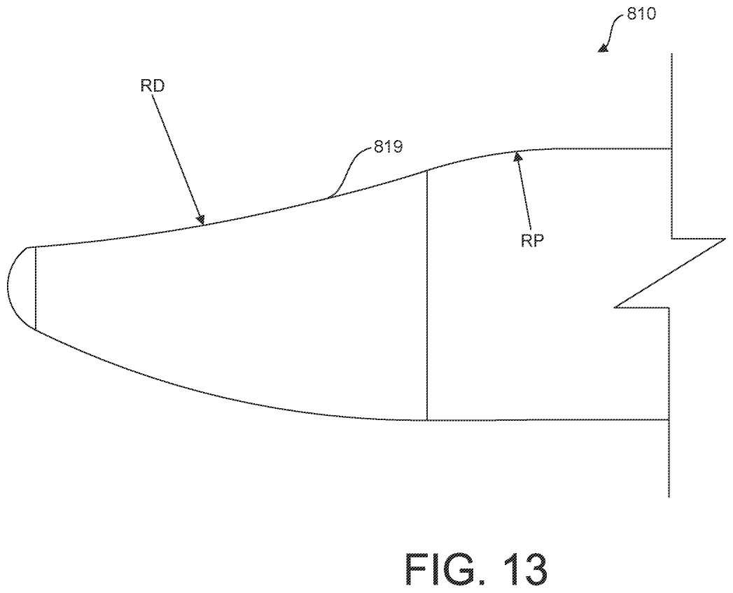

A group of specific but non-limiting alternate outer component window shapes are defined in FIGS. 7-13 for respective outer components 210, 310, 410, 510, 610, 710, 810. Each of the illustrated curvatures is labelled either “RD” for radius distal or “RP” for radius proximal. Relative radii positions, directions, and lengths are noted in association with each embodiment. In some embodiments, each radius of the outer component window is a precise radius of a fixed amount, but in other embodiments, the radii may be somewhat varied along their length to transition between two or more radii within a range. In FIG. 7, both RD and RP define concave segments of a window 219 in the outer component 210, and RP is relatively larger than RD. In another embodiments (not shown), RD may be relatively larger than RP or RP and RD may be substantially the same radius. In FIG. 8, RD defines a convex segment and RP defines a concave segment of a window 319 in the outer component 310, and RD and RP are substantially the same radius. In FIG. 9, RD defines a convex segment and RP defines a concave segment of a window 419 in the outer component 410, and RP has a relatively larger radius than RD. In FIG. 10, RD defines a convex segment and RP defines a concave segment of a window 519 in the outer component 510, and RD has a relatively larger radius than RP. In FIG. 11, RD defines a concave segment and RP defines a convex segment of a window 619 in the outer component 610, and RP has a relatively larger radius than RD. In FIG. 12, RD defines a concave segment and RP defines a convex segment of a window 719 in the outer component 710, and RD and RP are substantially the same radius. In FIG. 13, RD defines a concave segment and RP defines a convex segment of a window 819 in the outer component 810, and RD has a relatively larger radius than RP.

The inner component 120 shown in FIGS. 1-4 is sized to fit within the outer component 110. The inner component 120 is configured to be rotated relative to the outer component 110. In particular, the inner component 120 includes a torque transfer element 125 (FIG. 1) configured to engage with and be turned by the drive unit 101 (FIG. 14). The torque transfer element 125 extends from a proximal end of the inner component 120 to enable the inner component 120 to be turned by the drive unit 101 when the outer component 110 is seated in the drive unit 101, as shown in FIG. 14. Any effective mechanism for transferring torque from a drive unit to an inner component may be used in other embodiments. The inner component 120 shown is generally tubular. This configuration may be useful in removing material cut or otherwise manipulated by a cutting tool by applying a negative pressure to the pathway within the tubular inner component 120.

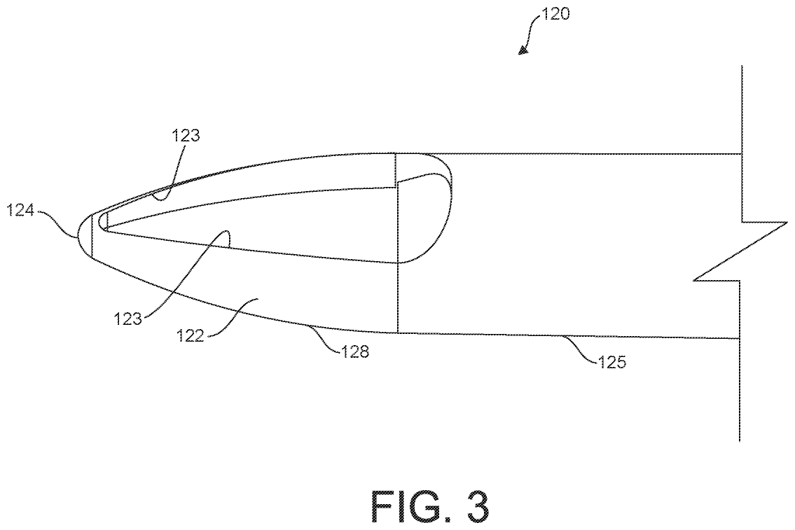

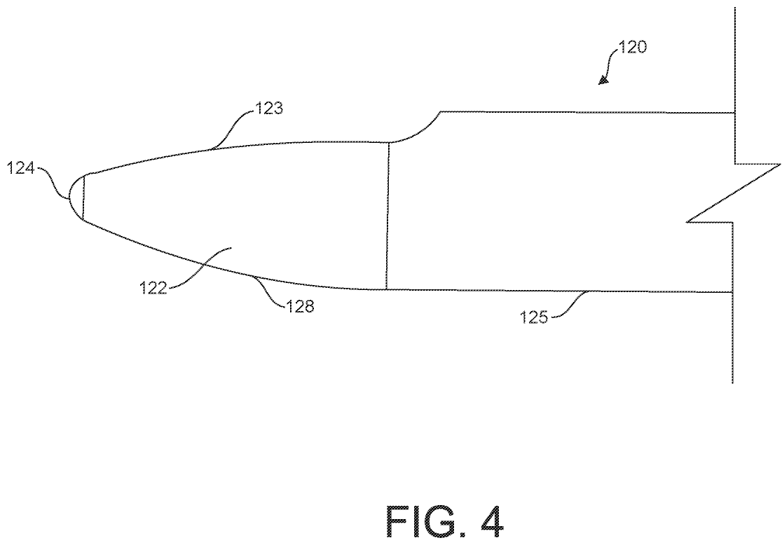

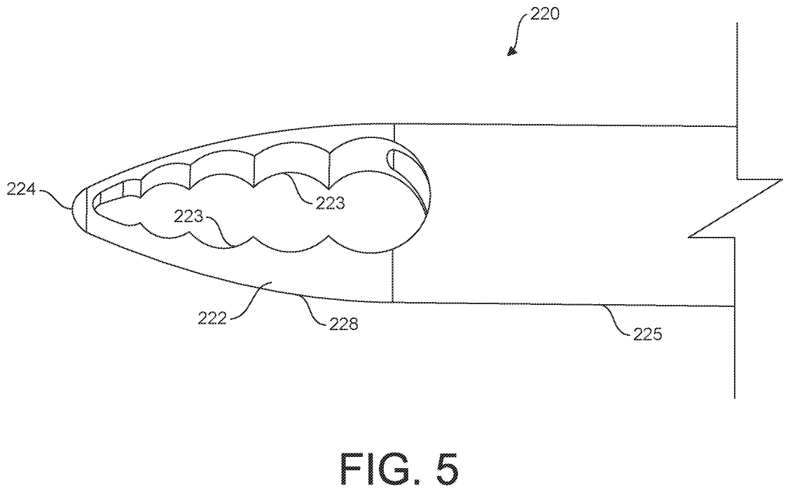

A distal end of the inner component 120 of the illustrated embodiment includes a cutter 122, as shown at two different angles of rotation in FIGS. 3 and 4. The cutter 122 may include one or more sharpened edges 123 that one or both slice tissue directly and work in combination with an inner edge of the outer component window 119 to shear tissue between the inner component 120 and the outer component 110. A sharpened edge of some embodiments may be the same edge that shears tissue in combination with an outer component. The cutter 122 is integral with a distal end of the inner component 120. In other embodiments, a cutter may be a module or component configured to couple at a distal end of the inner member by any effective mechanism. An alternate embodiment of an inner component 220 with a cutter 222 and sharpened edges 223 is illustrated in FIG. 5. The sharpened edges 223 include serrations or teeth that may be more effective in removing certain types of tissue and in certain circumstances. Cutting elements of inner components 120, 220 may be formed from the same material as a rotatable shaft or a different material. Cutting elements of various embodiments may include blades, burrs, rasps, abrasives, or any other devices effective to cut, abrade, scratch, rub, dislodge, or otherwise manipulate tissue.

The inner components 120, 220 depicted in FIGS. 3-5 include a cylindrical portion 125, 225 and an ogive-shaped distal portion 128, 228 coupled to the cylindrical portion 125, 225. The meanings of an “ogive” or “ogive-shaped” portion are substantially the same as the meanings provided herein in association with the outer component 110. As shown in FIGS. 3-5, a far distal tip 124, 224 of the inner components 120, 220 has a closed generally spherical tip at a cross-sectional center of the inner components 120, 220. Other embodiments may include other shapes that are configured to rotate effectively against or adjacent to an interior end portion of an outer component 110, 210, 310, 410, 510, 610, 710, 810 or with a bearing.

Embodiments of the cutting tool may also include a drive unit coupled to an outer component of the cutting tool and coupled to an inner component of the cutting tool. Such drive unit embodiments are capable of turning the inner component relative to the outer component. Drive units of some embodiments are motorized. Other embodiments may be manually moved or may be actuated by any other effective mechanism. The cutting tool 100 depicted in FIGS. 1 and 14 may include the drive unit 101 coupled to the base 111 of the outer component 110, as specifically described herein. The cutting tool 100 is also coupled to the inner component 120 through the torque transfer element 125 that extends from a proximal end of the inner component 120. Activation of the drive unit 101 of some embodiments will enable rotation of the inner component 120 relative to the outer component 110. Function and control of the drive unit 101 in combination with the other components of the cutting tool 100 are illustrated in a system diagram in FIG. 14. Drive units of some embodiments may include a processor coupled with a power control. These elements may be electrically coupled with such elements as a revolution rate selector, a revolution direction selector, alert controls, and an indicator. The alert control may be used to one or both select and indicate whether an alert is to be provided with a lighted indication, an audible indication, a vibratory indication, or any other effective indication. The indicator may be used in association with one or more of the other functions of a drive unit. For example and without limitation, the indicator may be used to set or display a speed or direction of rotation for the device, may be used as a power indicator, or may be used for one or a combination of these or other purposes. Control and display functions may be accomplished with hardwired or so-called “soft” programmable function buttons or keys that are part of a drive unit. The system may further include display screens of various types. These and other function and control mechanisms may alternatively or in combination be performed by an external control system.

A control system is illustrated in FIG. 14 coupled with the drive unit 101, which is couple with the cutting tool 100 as part of a system for cutting 1. The system for cutting 1 illustrated in FIG. 14 therefore includes a cutting tool 100 with a drive unit 101, a control system with a resection control 1510, and a fluid management system. The illustrated control system includes the resection control 1510 that is electrically coupled with the drive unit 101 by a cable 1511. The resection control 1510 may be used to one or more of provide power to the drive unit 101, receive operator inputs from the drive unit 101, sense operating parameters of the drive unit 101, receive operator inputs from external switches or controls such as foot operated switches or controls, provide, set, or display alerts to a user based on operations of the cutting tool 100, and send and receive signals to and from the pump control 1520. The resection control 1510 illustrated also includes a resection display panel 1518, which may be used to communicate information to a user and may be used to input settings or other information into the resection control 1510 or other connected components of the control system. Other knobs, switches, controls, and the like may be used to control, set, or calibrated the resection control 1510 as well.

In the illustrated embodiment, the pump control 1520 is part of a fluid management system used in conjunction with fluid supply, tubing, and disposal components as described herein to facilitate the use of the cutting tool 100. For example and without limitation, fluids such as saline may be used during endoscopic or arthroscopic surgical procedures to provide a clear operating medium in which to perform surgical tasks. The pump control 1520 may be used to one or more provide fluid to the drive unit 101, sense operating parameters of the drive unit 101, manage waste fluid, receive operator inputs from external switches or controls such as foot operated switches or controls, and send and receive signals to and from the resection control 1510. A fluid inflow line 1521 is shown coupled between the pump control 1520 and a patient joint cannula 1522. The patient joint cannula 1522 may provide one or both a passageway through which the cutting tool 100 may be introduced into a joint and an entry port for fluid supplied though the fluid inflow line 1521. In other embodiments, one or more additional fluid lines may be used to supply fluid or remove fluid from a surgical site from locations different than those illustrated. A saline bag 1523 is shown providing a fresh fluid supply to the pump control 1520 through a supply line 1524 in the present embodiment. Any other effective fluid source may be used in various embodiments. A suction line 1525 is shown coupled between the cutting tool 100 and the pump control 1520, which when activated draws waste fluid through the cutting tool 100 and into the pump control 1520 where the fluid may be diverted for waste removal. A waste line 1527 is shown coupled between the pump control 1520 and a waste receptacle 1529. Any other effective supply or waste handling mechanisms may be used in other embodiments. The pump control 1520 illustrated also includes a pump control display panel 1528, which may be used to communicate information to a user and may be used to input settings or other information into the pump control 1520 or other connected components of the control system. Other knobs, switches, controls, and the like may be used to control, set, or calibrated the pump control 1520 as well.

Another embodiment of the invention is a method of removing tissue with a system for cutting, such as the system for cutting 1. Acts of the method may include advancing a cutting tool 100 with an outer component 110, 210, 310, 410, 510, 610, 710, 810 having a bullet-shaped distal end and an outer component window 119, 219, 319, 419, 519, 619, 719, 819 into tissue, wherein the outer component window at least in part includes a curvature relative to a longitudinal axis of the outer component. In some embodiments, the curvature relative to the longitudinal axis is a concave curvature or includes a concave curvature, as for example illustrated in FIGS. 1, 2 and 6-13. In some embodiments, the outer component window curvature includes a first concavely curved portion with a first radius and a second concavely curved portion with a second radius that is larger than the first radius (FIG. 7). In different embodiments, the first radius may be proximal of the second radius or the first radius may be distal of the second radius. The first and second radii may be substantially equal in some instances. In some embodiments, the outer component window curvature includes at least a concavely curved portion and a convexly curved portion (FIGS. 8-12). The concavely curved portion may be more distal (FIGS. 11-13), or the convexly curved portion may be more distal (FIGS. 8-10). The radius of the concavely curved portion may greater than the radius of the convexly curved portion (FIGS. 9 and 13), or the radius of the concavely curved portion may be less than the radius of the convexly curved portion (FIGS. 10 and 11). In some embodiments, the radii of the concavely curved portion and the convexly curved portion are substantially the same (FIGS. 8 and 12).

The method of removing tissue with a system for cutting may also include the act of injecting fluid into a surgical site under control of a fluid management system. This act may include injecting fluid supplied from the saline bag 1523 to the pump control 1520 through a supply line 1524 and to surgical site through the fluid inflow line 1521 and into a patient joint cannula 1522. The patient joint cannula 1522 may provide one or both a passageway through which the cutting tool 100 may be introduced into a joint and an entry port for fluid supplied though the fluid inflow line 1521.

Method embodiments may also include rotating an inner component 120, 220 sized to fit within a central hollow portion of the outer component 110, 210, 310, 410, 510, 610, 710, 810 relative to the outer component under control of a resection control 1510 such that a portion of the inner component 120, 220 intermittently extends through the outer component window 119, 219, 319, 419, 519, 619, 719, 819, as for example is shown in FIG. 2, to shear and remove tissue that is adjacent to the cutting tool 100.

Various embodiments of a system wholly or its components individually may be made from any biocompatible material. For example and without limitation, biocompatible materials may include in whole or in part: non-reinforced polymers, reinforced polymers, metals, ceramics, adhesives, reinforced adhesives, and combinations of these materials. Reinforcing of polymers may be accomplished with carbon, metal, or glass or any other effective material. Examples of biocompatible polymer materials include polyamide base resins, polyethylene, Ultra High Molecular Weight (UHMW) polyethylene, low density polyethylene, polymethylmethacrylate (PMMA), polyetheretherketone (PEEK), polyetherketoneketone (PEKK), a polymeric hydroxyethylmethacrylate (PHEMA), and polyurethane, any of which may be reinforced. Example biocompatible metals include stainless steel and other steel alloys, cobalt chrome alloys, zirconium, oxidized zirconium, tantalum, titanium, titanium alloys, titanium-nickel alloys such as Nitinol and other superelastic or shape-memory metal alloys.

Terms such as proximal, distal, near, far, and the like have been used relatively herein. However, such terms are not limited to specific coordinate orientations, distances, or sizes, but are used to describe relative positions referencing particular embodiments. Such terms are not generally limiting to the scope of the claims made herein. Any embodiment or feature of any section, portion, or any other component shown or particularly described in relation to various embodiments of similar sections, portions, or components herein may be interchangeably applied to any other similar embodiment or feature shown or described herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A cutting tool comprising:
an outer component having a longitudinal axis, the outer component including a cylindrical portion, an ogive-shaped distal portion coupled to the cylindrical portion, and an outer component window into a central hollow portion in the outer component; and
an inner component sized to fit within the central hollow portion of the outer component and be rotated relative to the outer component;
wherein the outer component window at least in part includes a curvature relative to the longitudinal axis of the outer component.

2. The cutting tool of claim 1 wherein the ogive-shaped distal portion comprises at least one of the following shapes: secant ogive, spherically blunted secant ogive, tangent ogive, spitzer ogive, spherically blunted tangent ogive, elliptical ogive, conic ogive, spherically blunted conic ogive, bi-conic ogive, spherically blunted bi-conic ogive, parabolic series ogives, power series ogives, and Haack series ogives.

3. The cutting tool of claim 1 wherein the outer component includes a closed distal tip at a cross-sectional center of the outer component.

4. The cutting tool of claim 1 wherein the outer component window curvature is in both the cylindrical portion and the ogive-shaped distal portion.

5. The cutting tool of claim 1 wherein the outer component window curvature includes a first concavely curved portion with a first radius and a second concavely curved portion with a second radius that is larger than the first radius.

6. The cutting tool of claim 5 wherein the first concavely curved portion is proximal of the second concavely curved portion.

7. The cutting tool of claim 5 wherein the first concavely curved portion is distal of the second concavely curved portion.

8. The cutting tool of claim 1 wherein the outer component window curvature includes a concavely curved portion and a convexly curved portion.

9. The cutting tool of claim 8 wherein the concavely curved portion is proximal of the convexly curved portion.

10. The cutting tool of claim 8 wherein the concavely curved portion is distal of the convexly curved portion.

11. The cutting tool of claim 8 wherein the radius of the concavely curved portion is greater than the radius of the convexly curved portion.

12. The cutting tool of claim 8 wherein the radius of the concavely curved portion is less than the radius of the convexly curved portion.

13. The cutting tool of claim 1 wherein the inner component is generally tubular.

14. The cutting tool of claim 1 wherein the inner component includes a closed distal tip at a cross-sectional center of the inner component.

15. The cutting tool of claim 1 wherein the inner component includes an inner component cylindrical portion, an inner component ogive-shaped distal portion coupled to the inner component cylindrical portion, and an inner component window into a central hollow portion in the inner component.

16. The cutting tool of claim 15 wherein inner component window includes a sharpened surface configured to shear material between the sharpened surface and the outer component.

17. The cutting tool of claim 16 wherein the sharpened surface includes serrations.

18. A system for cutting comprising:
a cutting tool comprising:
an outer component having a longitudinal axis, the outer component including a cylindrical portion, an ogive-shaped distal portion coupled to the cylindrical portion, and an outer component window into a central hollow portion in the outer component,
an inner component sized to fit within the central hollow portion of the outer component and be rotated relative to the outer component, and
a drive unit coupled to the outer component and coupled to the inner component and capable of rotating the inner component relative to the outer component,
wherein the outer component window at least in part includes a curvature relative to the longitudinal axis of the outer component;
a resection control electrically coupled to the cutting tool, the resection control configured to provide control signals to the cutting tool; and
a fluid management system comprising a pump control and a fluid passage between the pump control and a surgical site, the fluid management system configured to control a fluid supply and to suction fluid through the cutting tool.

19. The system for cutting of claim 18 wherein the ogive-shaped distal portion comprises at least one of the following shapes: secant ogive, spherically blunted secant ogive, tangent ogive, spitzer ogive, spherically blunted tangent ogive, elliptical ogive, conic ogive, spherically blunted conic ogive, bi-conic ogive, spherically blunted bi-conic ogive, parabolic series ogives, power series ogives, and Haack series ogives.

20. The system for cutting of claim 18 wherein the outer component includes a closed distal tip at a cross-sectional center of the outer component.

21. The system for cutting of claim 18 wherein the outer component window curvature is in both the cylindrical portion and the ogive-shaped distal portion.

22. The system for cutting of claim 18 wherein the outer component window curvature includes a first concavely curved portion with a first radius and a second concavely curved portion with a second radius that is larger than the first radius.

23. The system for cutting of claim 22 wherein the first concavely curved portion is proximal of the second concavely curved portion.

24. The system for cutting of claim 22 wherein the first concavely curved portion is distal of the second concavely curved portion.

25. The system for cutting of claim 18 wherein the outer component window curvature includes a concavely curved portion and a convexly curved portion.

26. The system for cutting of claim 25 wherein the concavely curved portion is proximal of the convexly curved portion.

27. The system for cutting of claim 25 wherein the concavely curved portion is distal of the convexly curved portion.

28. The system for cutting of claim 25 wherein the radius of the concavely curved portion is greater than the radius of the convexly curved portion.

29. The system for cutting of claim 25 wherein the radius of the concavely curved portion is less than the radius of the convexly curved portion.

30. The system for cutting of claim 18 wherein the inner component is generally tubular.

31. The system for cutting of claim 18 wherein the inner component includes a closed distal tip at a cross-sectional center of the inner component.

32. The system for cutting of claim 18 wherein the inner component includes an inner component cylindrical portion, an inner component ogive-shaped distal portion coupled to the inner component cylindrical portion, and an inner component window into a central hollow portion in the inner component.

33. The system for cutting of claim 32 wherein inner component window includes a sharpened surface configured to shear material between the sharpened surface and the outer component.

34. The system for cutting of claim 33 wherein the sharpened surface includes serrations.

35. A method of removing tissue with a system for cutting comprising:
advancing a cutting tool with an outer component having a bullet-shaped distal end and an outer component window into tissue, wherein the outer component window at least in part includes a curvature relative to a longitudinal axis of the outer component;
injecting fluid into a surgical site and suctioning fluid through the cutting tool under control of a fluid management system;
rotating an inner component sized to fit within a central hollow portion of the outer component relative to the outer component under control of a resection control such that a portion of the inner component intermittently extends through the outer component window to shear and remove tissue that is adjacent to the cutting tool.

36. The method of claim 35 wherein advancing a cutting tool with an outer component window that at least in part includes a curvature relative to the longitudinal axis of the outer component, includes advancing a cutting tool where the outer component window curvature includes a first concavely curved portion with a first radius and a second concavely curved portion with a second radius that is larger than the first radius.

37. The method of claim 36 wherein advancing a cutting tool includes advancing a cutting tool wherein the first concavely curved portion is proximal of the second concavely curved portion.

38. The method of claim 36 wherein advancing a cutting tool includes advancing a cutting tool wherein the first concavely curved portion is distal of the second concavely curved portion.

39. The method of claim 35 wherein advancing a cutting tool with an outer component window that at least in part includes a curvature relative to the longitudinal axis of the outer component, includes advancing a cutting tool where the outer component window curvature includes a concavely curved portion and a convexly curved portion.

40. The method of claim 39 wherein advancing a cutting tool includes advancing a cutting tool wherein the concavely curved portion is proximal of the convexly curved portion.

41. The method of claim 39 wherein advancing a cutting tool includes advancing a cutting tool wherein the concavely curved portion is distal of the convexly curved portion.

42. The method of claim 39 wherein advancing a cutting tool includes advancing a cutting tool wherein the radius of the concavely curved portion is greater than the radius of the convexly curved portion.

43. The method of claim 39 wherein advancing a cutting tool includes advancing a cutting tool wherein the radius of the concavely curved portion is less than the radius of the convexly curved portion.
